(19) United States
(12) Patent Application Publication
(10) Pub. No.: US 2025/0268904 A1
(43) Pub. Date: Aug. 28, 2025

(54) THERAPEUTICS AGAINST PATHOGENIC CORONAVIRUSES

(71) Applicants: The USA, as represented by the Secretary, Department of Health and Human Services, Bethesda, MD (US); University of Maryland, Baltimore, Baltimore, MD (US)

(72) Inventors: Melvin L. DePamphilis, Olney, MD (US); Arup R. Chakraborty, Kolkata (IN); Matthew B. Frieman, Baltimore, MD (US)

(73) Assignees: The USA, as represented by the Secretary, Department of Health and Human Services, Bethesda, MD (US); University of Maryland, Baltimore, Baltimore, MD (US)

(21) Appl. No.: 18/028,490

(22) PCT Filed: Nov. 19, 2021
(86) PCT No.: PCT/US2021/060122

§ 371 Date: Mar. 24, 2023

Related U.S. Application Data
(60) Provisional application No. 63/119,522, filed on Nov. 30, 2020.

(51) Int. Cl. A61K 31/5377; A61P 31/14

(52) U.S. Cl. CPC A61K 31/5377; A61P 31/14

(57) ABSTRACT

Compounds for use in treating a coronavirus infection in a subject, particularly an infection caused by SARS-CoV-2, SARS-CoV or MER-CoV.

### CROSS REFERENCE TO RELATED APPLICATIONS

This claims the benefit of U.S. Provisional Application No. 63/119,522, filed Nov. 30, 2020, which is incorporated by reference herein.

### ACKNOWLEDGMENT OF GOVERNMENT SUPPORT

This invention was made with government support under ZIA HD000506 awarded by the National Institute of Child Health and Human Development. The government has certain rights in the invention.

### BACKGROUND

Severe acute respiratory syndrome coronavirus (SARS-CoV), Severe acute respiratory syndrome coronavirus 2 (SARS-CoV-2), and Middle East respiratory syndrome coronavirus (MERS-CoV) are three highly transmissible pathogenic coronaviruses that emerged in humans over the past 17 years. SARS-CoV-2 is responsible for the current COVID-19 pandemic that entered the USA in January 2020 and causes the disease termed COVID-19. As of November 2020, COVID-19 has infected ˜12 million people in the USA alone where it has accounted for ˜250,000 deaths, more than twice the number of US deaths from the wars in Korea, Vietnam, Iraq and Afghanistan combined.

Pathogenic coronaviruses SARS-CoV-2, MERS-CoV and SARS-CoV enter the body through the nose, mouth or eyes, then attach to cells in the airways through binding to their receptor, ACE2 for SARS-CoV and SARS-CoV-2; DPP4 for MERS-CoV. Coronaviruses infect the cell by fusing its viral envelope with the cell at either the plasma membrane or endosomal membrane. The strategy pursued herein is to focus on the fact that both coronavirus infection and replication depend on ‘membrane trafficking’, the cell's use of membrane-bound vesicles to transport materials throughout the cell, to internalize materials such as the virus from extracellular space, and to subsequently release newly replicated virus.

### SUMMARY

Disclosed herein are compounds for use in treating a coronavirus infection in a subject, particularly an infection caused by SARS-CoV-2, SARS-CoV or MER-CoV.

In one embodiment, there is disclosed a method of inhibiting a SARS-CoV-2 infection in a subject, comprising administering to the subject a therapeutically effective amount of a compound, or a pharmaceutically salt thereof, of formula (I):

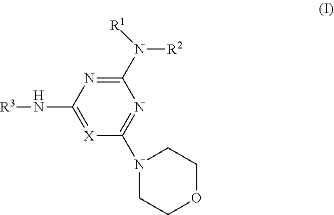

wherein R¹ and R² are independently H, C₁-C₆ alkyl, substituted C₁-C₆ alkyl, C₆-C₁₀ aryl, substituted C₆-C₁₀ aryl, or wherein R¹ and R² taken together with the N to which they are attached, form a 5- or 6-membered heterocyclyl ring;
R³ is C₆-C₁₀ aryl, optionally substituted C₆-C₁₀ aryl, R⁴CH═N— wherein R⁴ is C₆-C₁₀ aryl, heteroaryl, or fused bicyclic heteroaryl, or R⁴═CH—NH— wherein R⁴ is C₆-C₁₀ aryl, heteroaryl, or fused bicyclic heteroaryl; and
X is CH or N, provided that the compound of formula I is not vacuolin-1,
   thereby inhibiting the SARS-CoV-2 infection in the subject.

The foregoing will become more apparent from the following detailed description, which proceeds with reference to the accompanying figures.

### BRIEF DESCRIPTION OF THE DRAWINGS

FIG. 1. In vitro, WX8-family compounds bound primarily to PIKfyve and secondarily to PIP4K2C. The binding affinity of 10 μM WX8 was profiled against 468 human kinases by DiscoverX KINOMEscan (San Diego, CA). Compounds that bind to the active site of a protein kinase prevented the protein from binding to an immobilized active site ligand, thereby reducing the amount of protein captured on a solid support. Each kinase was tagged with a unique DNA sequence that allowed the amount of protein bound to the solid support to be quantified by PCR. The mean equilibrium dissociation constant (Kd) for each compound was then determined for the top three hits (PIKFYVE, PIP4K2C and MTOR) from two independent titration curves. A dissociation constant (Kd) was calculated by measuring the amount of kinase captured on the solid support as a function of the test compound concentration (nM) on a log₁₀ scale. Mean Kd values (±range) are given for each WX8-family member. The ratios of PIP4K2C/PIKfyve and mTOR/PIKfyve indicate the relative specificity of each compound for PIKfyve. Phosphatidylinositol (PI) contains two non-polar fatty acid tails consisting of 18 or 20 carbons linked to inositol that can be converted into various phosphatidylinositol phosphates.

FIG. 2. In vivo, WX8 bound primarily to the PIKfyve and secondarily to PIP4K2C. WX8 was profiled quantitatively against all the native kinases in melanoma A375 cells under conditions where protein-protein interactions, differential phosphorylation, and other naturally occurring modifications are preserved (KiNativ, http://www.kinativ.com/technology.htmi). Cells were cultured for one hour with either 1 μM or 0.05 μM WX8. The cells were then treated with biotinylated acyl phosphates of ATP and ADP irreversibly react with conserved lysine residues in the ATP-binding pocket of protein kinases, lipid kinases and heat shock proteins. Proteins were identified by mass spectroscopy. Results were analyzed using a melanoma A375-kinase-ATP probe list of 312 targets. The averages of three independent assays are reported (Student T-test score <0.04).

FIG. 3. PIKfyve inhibitors rapidly induced cytoplasmic vacuolation in an African Green monkey kidney cell line (Vero E6). Cells were plated at low seeding density and then cultured in the presence of vehicle control dimethylsulfoxide (DMSO), 1 μM WX8 or 1 μM NDF for 3 hours. Images were photographed at 10×.

FIG. 4. PIKfyve inhibitors suppressed Vero E6 cell proliferation, but they did not induce cell death. Vero E6 cells were plated at low density and then cultured overnight before adding WX8 at the concentrations indicated and then cultured for 3 days. (A) Attached cells were collected by trypsinization and counted. The fraction of cells (%) as well as the change in the total number of cells (fold change) relative to cells cultured in the presence of the WX8 vehicle control are given. (B) Attached and unattached cells were combined and stained with annexin-V to identify cells undergoing apoptosis and propidium iodide (PI) to identify dead cells that were permeable to a nuclear DNA stain. The cells were then analyzed by fluorescence activated cell sorting. Quadrant (Q) Q3 contained the unstained (live) cells. Q1 contained cells stained only with annexin-V (early apoptosis). Q2 contained cells stained with both annexin-V and PI (apoptotic dead cells). Q4 contained cells stained only with PI (necrotic dead cells). (C) Data were normalized to the fraction of live cells. Error bars indicate ±SEM. (D) Cells were first permeabilized, stained with PI and then analyzed by fluorescence activated cell sorting in order to quantify the fraction of cells with <2N DNA content. (E) The fraction of cells with <2N DNA content ±SEM.

FIGS. 5-7. Inhibition of PIKfyve protects mammalian cells from SARS-CoV-2 infection. Vero E6 cells were cultured overnight before being pretreated with WX8, NDF, WWL, or Apilimod, at the concentrations indicated for two hours prior to infection. Cells were then infected with SARS-CoV-2 expressing GFP at a multiplicity of infection of 0.1 pfu/cell. Cells were then cultured for 48 hours, followed by cell fixation, nuclear staining, and quantification of percent infected cells using a Nexcelon Celigo. The percent SARS-CoV-2 inhibition was then calculated (blue squares). Separate plates were also processed in the absence of virus to assess compound cytotoxicity (red squares), quantified by the loss of cellular ATP (Cell Titer Glo, Promega). Error bars indicate standard deviation.

### DETAILED DESCRIPTION

### Overview

To date, Remdesivir is the only FDA approved medication against COVID-19. Remdesivir inhibits the RNA-dependent RNA polymerase required to replicate the coronavirus RNA genome, but its effectiveness relative to placebo is marginal. Chloroquine and hydroxychloroquine are specific inhibitors of lysosomal activity that inhibit SARS-CoV-2 infection in cultured cells, but both drugs have dangerous side effects and neither drug has proven effective against COVID-19. Nevertheless, the effectiveness of chloroquine in inhibiting SARS-CoV-2 infection of cultured cells with little toxicity to the cells suggested that small molecules that inhibit lysosome homeostasis might exhibit therapeutic potential against pathogenic coronaviruses. Here we provide evidence that PIKfyve phosphatidylinositol kinase inhibitors exhibit therapeutic potential in preventing infection of mammalian cells by pathogenic coronaviruses.

PIKfyve converts phosphotidylinositol-3-phosphate into phosphatidylinositol-3,5-bisphosphate, a reaction that is essential for both lysosome homeostasis and early mammalian development. Five PIKfyve inhibitors (compounds 1-5 shown below) was discovered in a high-throughput screen for small molecules that induced excess DNA replication in cells derived from human cancers, but not in cells derived from normal tissues. These molecules were subsequently shown to disrupt multiple events in lysosome homeostasis and to inhibit PIKfyve with remarkable specificity. Two additional PIKfyve inhibitors, Vacuolin and Apilimod, were discovered and characterized independently.

Based on their chemical structures, these PIKfyve inhibitors can be classified into three subgroups: (A) those containing a 1,3,5-triazin-2-amine core with a morpholine adduct, (B) those containing a pyrimidine-4-amine core with a morpholine adduct and a benzaldehyde hydrazone adduct, and (C) those with two morpholine adducts attached to either a 1,3,5-triazin ring or a pyrimidine ring.

### Terminology

“Administration” as used herein is inclusive of administration by another person to the subject or self-administration by the subject.

The term “alkyl” means a straight-chain or branched alkyl substituent containing from, for example, 1 to about 6 carbon atoms, preferably from 1 to about 4 carbon atoms, more preferably from 1 to 2 carbon atoms. Examples of such substituents include methyl, ethyl, propyl, isopropyl, n-butyl, sec-butyl, isobutyl, tert-butyl, pentyl, isoamyl, hexyl, and the like.

The term “aryl” refers to an unsubstituted or substituted aromatic carbocyclic substituent, as commonly understood in the art, and the term “C6-C10 aryl” includes phenyl and naphthyl. It is understood that the term aryl applies to cyclic substituents that are planar and comprise 4n+2π electrons, according to Huckel's Rule.

Coronavirus: A family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future.

Non-limiting examples of betacoronaviruses include Middle East respiratory syndrome coronavirus (MERS-CoV), Severe Acute Respiratory Syndrome coronavirus (SARS-CoV) and SARS-CoV-2, Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

The viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

Coronavirus Disease 2019 (COVID-19): A disease caused by SARS-CoV-2 infection. Common symptoms include fever, cough, fatigue, shortness of breath or breathing difficulties, and loss of smell and taste. The incubation period may range from one to fourteen days. While most patients have mild symptoms, some develop acute respiratory distress syndrome (ARDS) possibly precipitated by cytokine storm, multi-organ failure, septic shock, and blood clots.

A host of underlying medical conditions are known to lead to increased risk of COVID-19, and severe symptoms of COVID-19, following infection with SARS-CoV-2. Non-limiting examples include heart disease, cancer, chronic obstructive pulmonary disease, type 2 diabetes, type 1 diabetes, obesity, chronic kidney disease, sickle cell disease, asthma, liver disease, chronic lung disease, high blood pressure, or a suppressed immune system due to medical treatment, infection with a pathogen other than SARS-CoV-2, or an autoimmune disorder.

The World Health Organization (WHO) has published testing guidelines for COVID-19 diagnosis (see, e.g., Laboratory Guidelines for the Detection and Diagnosis of COVID-19 virus infection, July 2020). The standard method of testing for SARS-CoV-2 infection is real-time reverse transcription polymerase chain reaction (RT-PCR) on respiratory samples obtained by a nasopharyngeal swab. Standard diagnostic methods of the detection of symptoms of COVID-19 is also utilized (e.g., lung inflammation, shortness of breath, low oxygen saturation, etc.)

Cytokine Storm Syndrome: A severe immune reaction in which the innate immune system causes uncontrolled and excessive release of cytokines into the blood. Excessive production of proinflammatory cytokines can aggravate existing respiratory distress, as well as cause overwhelming systemic inflammation, hemodynamic instability, multiple organ dysfunction, and potentially death. Cytokine storm syndrome is also called hypercytokinemia. Detection of cytokine storm syndrome in a patient can be accomplished using standard diagnostic methods, including but not limited to elevation of plasma C-reactive protein(CRP) levels, elevation of interleukin-6 (IL-6) levels, abnormalities of markers of blood clotting such as D-dimer or fibrinogen and elevated ferritin levels. Protocols for detecting cytokine storm in a COVID-19 patient are known and described, for example, in Soy et al., Clin Rheumatol., 39(7):2085-2094, 2020. Additional information concerning cytokine storm syndrome can be found, for example, in Ye et al., Journal of Infection, 80(6):607-613, 2020.

Dyspnea (shortness of breath): An intense tightening in the chest, air hunger, difficulty breathing, breathlessness or a feeling of suffocation.

The term “heterocyclyl,” as used herein, refers to a monocyclic or bicyclic 5- or 6-membered ring system containing one or more heteroatoms selected from the group consisting of O, N, S, and combinations thereof. The heterocyclyl group can be an aliphatic heterocyclyl group. The heterocyclyl group can be a monocyclic heterocyclyl group or a bicyclic heterocyclyl group. Suitable bicyclic heterocyclyl groups include monocylic heterocyclyl rings fused to a C6-C10 aryl ring, for example, dihydrobenzofuran or 1,2,3,4-tetrahydroquinoline, 1,2,3,4-tetrahydroisoquinoline, or indoline. Non-limiting examples of suitable heterocyclyl groups include tetrahydrofuranyl, tetrahydropyranyl, tetrahydrothiopheneyl, pyrrolidinyl, piperidinyl, and morpholinyl. The heterocyclyl group is optionally substituted with 1, 2, 3, 4, or 5 substituents as recited herein such as with alkyl groups such as methyl groups, ethyl groups, and the like, or with aryl groups such as phenyl groups, naphthyl groups and the like, wherein the aryl groups can be further substituted with, for example halo, dihaloalkyl, trihaloalkyl, nitro, hydroxy, alkoxy, aryloxy, amino, substituted amino, alkylcarbonyl, alkoxycarbonyl, arylcarbonyl, aryloxycarbonyl, thio, alkylthio, arylthio, and the like, wherein the optional substituent can be present at any open position on the heterocyclyl group.

The term “heteroaryl” refers to a monocyclic or bicyclic 5- or 6-membered ring system as described herein, wherein the heteroaryl group is unsaturated and satisfies Hulckel's rule. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, pyridazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heterocyclyl or heteroaryl group is optionally substituted with 1, 2, 3, 4, or 5 substituents as recited herein such as with alkyl groups such as methyl groups, ethyl groups, and the like, halo groups such as chloro, or hydroxyl groups, with aryl groups such as phenyl groups, naphthyl groups and the like, wherein the aryl groups can be further substituted with, for example halo, dihaloalkyl, trihaloalkyl, nitro, hydroxy, alkoxy, aryloxy, amino, substituted amino, alkylcarbonyl, alkoxycarbonyl, arylcarbonyl, aryloxycarbonyl, thio, alkylthio, arylthio, and the like, wherein the optional substituent can be present at any open position on the heterocyclyl or heteroaryl group, or with benzo groups, to form a group of, for example, benzofuran or indolyl.

Hypoxia: A condition in which the body or a region of the body is deprived of adequate oxygen.

In acute or silent hypoxia, a subject's oxygen level in blood cells and tissue can drop without any initial warning, even though the individual's chest x-ray shows diffuse pneumonia with an oxygen level below normal. Silent hypoxia has been reported in subject with COVID-19 who did not experience shortness of breath or coughing until their oxygen levels had plummeted to such a degree that the patients risked acute respiratory distress (ARDS) and organ failure.

Blood saturation levels of oxygen can be easily and non-invasively detected using pulse oximeters. Oxygen saturation in healthy patients range from 95-100%; hypoxia is a saturation level below 95%, see Teo, J. Med. Sys. 44: 134, 2020.

Inhibiting or treating a disease: Inhibiting the full development of a disease or condition, for example, in a subject who is at risk for a disease such as a SARS-CoV-2 infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents and the like, for example sodium acetate or sorbitan monolaurate. In particular embodiments, suitable for administration to a subject the carrier may be sterile, and/or suspended or otherwise contained in a unit dosage form containing one or more measured doses of the composition suitable to induce the desired immune response. It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

The phrase “pharmaceutically acceptable salt” is intended to include non-toxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media such as ether, ethyl acetate, ethanol, isopropanol, or acetonitrile are preferred.

Suitable bases include inorganic bases such as alkali and alkaline earth metal bases, such as those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of the compounds have an acidic moiety include sodium and potassium salts. Preferred pharmaceutically acceptable salts of the compounds have a basic moiety (such as a dimethylaminoalkyl group) include hydrochloride and hydrobromide salts. The compounds containing an acidic or basic moiety are useful in the form of the free base or acid or in the form of a pharmaceutically acceptable salt thereof.

It should be recognized that the particular counterion forming a part of any salt of the compound is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

Prodrugs of the disclosed compounds also are contemplated herein. A prodrug is an active or inactive compound that is modified chemically through in vivo physiological action, such as hydrolysis, metabolism and the like, into an active compound following administration of the prodrug to a subject. The term “prodrug” as used throughout this text means the pharmacologically acceptable derivatives such as esters, amides and phosphates, such that the resulting in vivo biotransformation product of the derivative is the active drug as defined in the compounds described herein. Prodrugs preferably have excellent aqueous solubility, increased bioavailability and are readily metabolized into the active inhibitors in vivo. Prodrugs of a compounds described herein may be prepared by modifying functional groups present in the compound in such a way that the modifications are cleaved, either by routine manipulation or in vivo, to the parent compound. The suitability and techniques involved in making and using prodrugs are well known by those skilled in the art. F or a general discussion of prodrugs involving esters see Svensson and Tunek, Drug Metabolism Reviews 165 (1988) and Bundgaard, Design of Prodrugs, Elsevier (1985).

The term “prodrug” also is intended to include any covalently bonded carriers that release an active parent drug of the present invention in vivo when the prodrug is administered to a subject. Since prodrugs often have enhanced properties relative to the active agent pharmaceutical, such as, solubility and bioavailability, the compounds disclosed herein can be delivered in prodrug form. Thus, also contemplated are prodrugs of the presently disclosed compounds, methods of delivering prodrugs and compositions containing such prodrugs. Prodrugs of the disclosed compounds typically are prepared by modifying one or more functional groups present in the compound in such a way that the modifications are cleaved, either in routine manipulation or in vivo, to yield the parent compound. Prodrugs may include compounds having a phosphonate, hydroxy, thio and/or amino group functionalized with any group that is cleaved in vivo to yield the corresponding amino, hydroxy, thio and/or phosphonate group, respectively. Examples of prodrugs can include, without limitation, compounds having an acylated amino group and/or a phosphonate ester or phosphonate amide group.

Pulmonary function: The function of the respiratory system, which can be measured through a variety of tests, including, but not limited to measurements of airflow (e.g. spirometry) or arterial blood gases (for example, oxygen saturation level, such as SpO2). Measurements of airflow included airflow rate, peak expiratory flow rate (PEFR), forced expiratory volume in the first second (FEV₁), and maximal midexpiratory rate (MMEFR).

SARS-CoV-2: SARS-CoV-2 is a positive-sense, single stranded RNA virus of the genus betacoronavirus that has emerged as a highly fatal cause of severe acute respiratory infection. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (N) - 3′. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-CoV-2 infection, including but not limited to, assessment of patient symptoms, background and genetic tests such as reverse transcription-polymerase chain reaction (RT-PCR), and antibody tests. The test can be done on patient samples such as respiratory or blood samples.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a camel or a bat. The subject can be a domestic animal (such as a dog or a cat) or a farm animal (such as a cow or a pig). In an additional example, a subject is selected that is in need of inhibiting of a coronavirus infection, such as a SARS-CoV-2 infection. For example, the subject is either uninfected and at risk of the coronavirus infection or is infected and in need of treatment.

“Substituted” or “substitution” refers to replacement of a hydrogen atom of a molecule or an R-group with one or more additional R-groups. Unless otherwise defined, the term “optionally-substituted” or “optional substituent” as used herein refers to a group which may or may not be further substituted with 1, 2, 3, 4 or more groups, preferably 1, 2 or 3, more preferably 1 or 2 groups. The substituents may be selected, for example, from C₁₋₆alkyl, C₂₋₆alkenyl, C₂₋₆alkynyl, C₃₋₈cycloalkyl, hydroxyl, oxo, C₁₋₆alkoxy, aryloxy, C₁₋₆alkoxyaryl, halo, C₁₋₆alkylhalo (such as CF₃ and CHF₂), C₁₋₆alkoxyhalo (such as OCF₃ and OCHF₂), carboxyl, esters, cyano, nitro, amino, substituted amino, disubstituted amino, acyl, ketones, amides, aminoacyl, substituted amides, disubstituted amides, thiol, alkylthio, thioxo, sulfates, sulfonates, sulfinyl, substituted sulfinyl, sulfonyl, substituted sulfonyl, sulfonylamides, substituted sulfonamides, disubstituted sulfonamides, aryl, arC₁₋₆alkyl, heterocyclyl and heteroaryl wherein each alkyl, alkenyl, alkynyl, cycloalkyl, aryl and heterocyclyl and groups containing them may be further optionally substituted. Optional substituents in the case N-heterocycles may also include but are not limited to C₁₋₆alkyl i.e. N—C₁₋₃alkyl, more preferably methyl particularly N-methyl.

Therapeutically effective amount: The amount of compound that is sufficient to prevent, treat, inhibit and/or ameliorate the symptoms and/or underlying causes of a disorder or disease, for example to prevent, inhibit, and/or treat a coronavirus infection, such as a SARS-CoV-2 infection. A therapeutically effective amount can be sufficient to reduce or eliminate a symptom of a disease, such as a SARS-CoV-2 infection. For instance, this can be the amount necessary to inhibit or prevent viral replication or to measurably alter outward symptoms of the viral infection, such as fever, cough, or difficulty breathing. In general, this amount will be sufficient to measurably inhibit virus replication or infectivity.

In one example, a desired response is to inhibit or reduce or prevent a SARS-CoV-2 infection. The SARS-CoV-2 infection does not need to be completely eliminated or reduced or prevented for the method to be effective. For example, administration of a therapeutically effective amount of the agent can decrease the SARS-CoV-2 infection (for example, as measured by infection of cells, or by number or percentage of subjects infected by SARS-CoV-2) by a desired amount, for example by at least 10%, at least 20%, at least 50%, at least 60%, at least 70%, at least 80%, at least 90%, at least 95%, at least 98%, or even at least 100% (elimination or prevention of detectable coronavirus infection, as compared to a suitable control.

A therapeutically effective amount of a compound can be administered in a single dose, or in several doses, for example daily, during a course of treatment. However, the therapeutically effective amount can depend on the subject being treated, the severity and type of the condition being treated, and the manner of administration. A unit dosage form of the compound can be packaged in a therapeutic amount, or in multiples of the therapeutic amount, for example, in a vial (e.g., with a pierceable lid) or syringe having sterile components.

The presently disclosed compounds can have at least one asymmetric center or geometric center, cis-trans center(C═C, C═N). All chiral, diasteromeric, racemic, meso, rotational and geometric isomers of the structures are intended unless otherwise specified. The compounds can be isolated as a single isomer or as mixture of isomers. All tautomers of the compounds are also considered part of the disclosure. The presently disclosed compounds also include all isotopes of atoms present in the compounds, which can include, but are not limited to, deuterium, tritium, ¹⁸F, etc.

In any of the above embodiments, the compound or salt can exist in one or more tautomeric forms. The term “tautomer” as used herein includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol; amide-to-imide; lactam-to-lactim; enamine-to-imine; and enamine-to-(a different) enamine tautomerizations. In an example, when R³ of formula (I) is a group of the formula: R⁴CH═N— and R⁴ includes a CH group bonded to the CH of R⁴CH═N—, such as —CH—CH═N—, a tautomer can be represented by the formula: —C═CH—NH—. Thus, the following structural representations are tautomeric to each other:

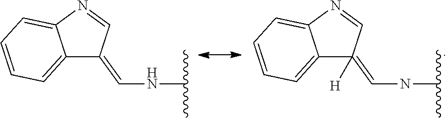

### Compounds, Pharmaceutical Compositions, and Methods of Use

The compounds disclosed herein for use in treating a coronavirus infection in a subject, particularly an infection caused by SARS-CoV-2, SARS-CoV or MER-CoV, are compounds, or pharmaceutically acceptable salts thereof, of formula (I):

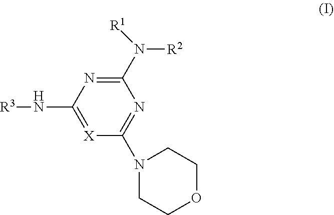

wherein R¹ and R² are independently H, C₁-C₆ alkyl, substituted C₁-C₆ alkyl, C₆-C₁₀ aryl, substituted C₆-C₁₀ aryl, or wherein R¹ and R² taken together with the N to which they are attached, form a 5- or 6-membered heterocyclyl ring;
R³ is C₆-C₁₀ aryl, substituted C₆-C₁₀ aryl, R⁴CH═N— wherein R⁴ is C₆-C₁₀ aryl, heteroaryl, or fused bicyclic heteroaryl, or R⁴═CH—NH— wherein R⁴ is C₆-C₁₀ aryl, heteroaryl, or fused bicyclic heteroaryl; and
X is CH or N.

In certain embodiments, the compound of formula I is not vacuolin-1.

In certain embodiments, X is N.

In certain embodiments, R¹ and R² taken together with the N to which they are attached, form morpholinyl.

In certain embodiments, at least one of R¹ or R² is H.

In certain embodiments, one of R¹ or R² is H, and the other of R¹ or R² is phenyl or substituted phenyl.

In certain embodiments, R³ is:

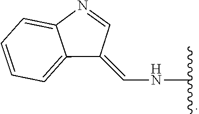 [SMILES: *N/C=C1\C=Nc2ccccc21]

In certain embodiments, R³ is phenyl or substituted phenyl. In certain embodiments, the phenyl is substituted with at least one substituent selected from an alkyl or a halogen.

In certain embodiments, R³ is —N═CH—R⁵, wherein R⁵ is phenyl or substituted phenyl. In certain embodiments, the phenyl is substituted with at least one substituent selected from an alkyl or a halogen.

In certain embodiments, the compound is

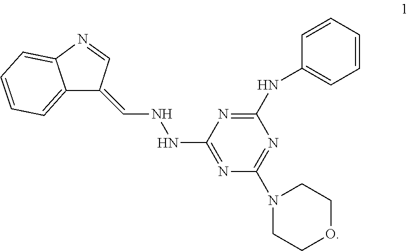 [SMILES: C1=Nc2ccccc2/C1=C/NNc1nc(Nc2ccccc2)nc(N2CCOCC2)n1]

(also referred to herein as compound WX8),

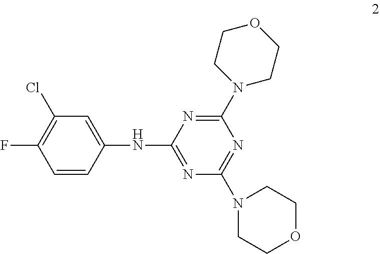 [SMILES: Fc1ccc(Nc2nc(N3CCOCC3)nc(N3CCOCC3)n2)cc1Cl]

(also referred to herein as compound XBA),

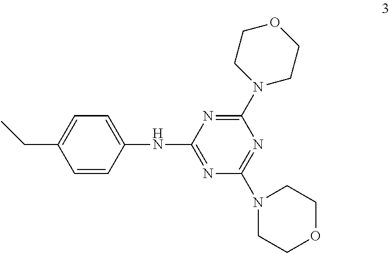 [SMILES: CCc1ccc(Nc2nc(N3CCOCC3)nc(N3CCOCC3)n2)cc1]

(also referred to herein as compound XB6),

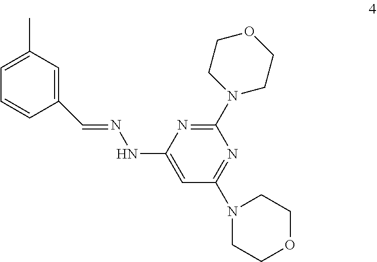 [SMILES: Cc1cccc(/C=N/Nc2cc(N3CCOCC3)nc(N3CCOCC3)n2)c1]

(also referred to herein as compound NDF),
or

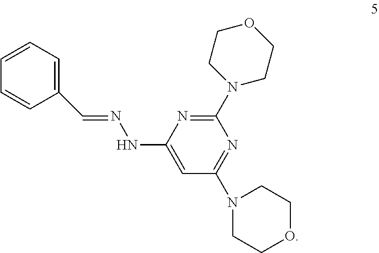 [SMILES: C(=N\Nc1cc(N2CCOCC2)nc(N2CCOCC2)n1)/c1ccccc1]

(also referred to herein as compound WWL).

The structures of vacuolin and apilimod are shown below:

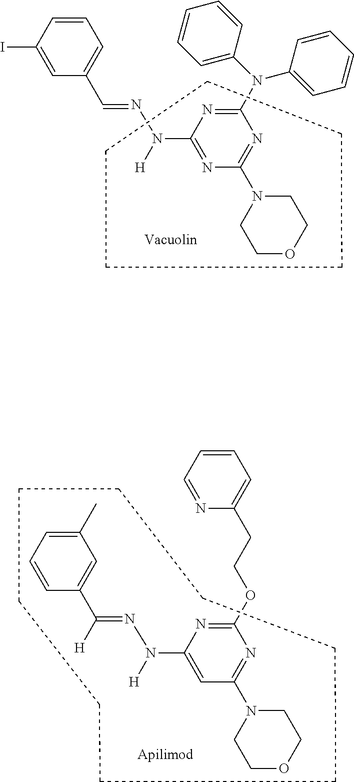

I. Compounds disclosed herein rapidly disrupt three events in lysosome homeostasis in a manner that is compound dependent, concentration dependent, time dependent and reversible.
A. They inhibit lysosome fission via tubulation without preventing homotypic lysosome fusion, thereby inducing accumulation of enlarged lysosomes, and preventing lysosome turnover.
B. They impaired trafficking of molecules into lysosomes without altering lysosomal acidity, thereby disrupting lysosomal function.
C. They inhibit heterotypic fusion between lysosomes and autophagosomes, thereby blocking autophagic flux.

IL Compounds disclosed herein bind specifically to PIKFYVE (phosphoinositide kinase FYVE-type zinc finger containing) and inhibit its activity.

In certain embodiments, the compounds disclosed herein can prevent SARS-CoV-2, the virus responsible for COVID-19 disease, from infecting mammalian cells in culture under conditions that have no detectable effect on the viability of normal cells. These compounds are effective at concentrations from 100 to 1000-times less than are toxic to uninfected cells.

In some embodiments, the methods disclosed herein involve administering to a subject in need of treatment a pharmaceutical composition, for example a composition that includes a pharmaceutically acceptable carrier and a therapeutically effective amount of one or more of the compounds disclosed herein. The compounds may be administered orally, parenterally (including subcutaneous injections (SC or depo-SC), intravenous (IV), intramuscular (IM or depo-IM), intrasternal injection or infusion techniques), sublingually, intranasally (inhalation), intrathecally, topically, ophthalmically, or rectally. The pharmaceutical composition may be administered in dosage unit formulations containing conventional non-toxic pharmaceutically acceptable carriers, adjuvants, and/or vehicles. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

In some embodiments, one or more of the disclosed compounds (including compounds linked to a detectable label or cargo moiety) are mixed or combined with a suitable pharmaceutically acceptable carrier to prepare a pharmaceutical composition. Pharmaceutical carriers or vehicles suitable for administration of the compounds provided herein include any such carriers known to be suitable for the particular mode of administration. Remington: The Science and Practice of Pharmacy, The University of the Sciences in Philadelphia, Editor, Lippincott, Williams, & Wilkins, Philadelphia, PA, 21^{st} Edition (2005), describes exemplary compositions and formulations suitable for pharmaceutical delivery of the compounds disclosed herein. In addition, the compounds may be formulated as the sole pharmaceutically active ingredient in the composition or may be combined with other active ingredients.

Upon mixing or addition of the compound(s) to a pharmaceutically acceptable carrier, the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. Where the compounds exhibit insufficient solubility, methods for solubilizing may be used. Such methods are known and include, but are not limited to, using cosolvents such as DMSO, using surfactants such as Tween®, and dissolution in aqueous sodium bicarbonate. Derivatives of the compounds, such as salts or prodrugs may also be used in formulating effective pharmaceutical compositions. The disclosed compounds may also be prepared with carriers that protect them against rapid elimination from the body, such as time-release formulations or coatings. Such carriers include controlled release formulations, such as, but not limited to, microencapsulated delivery systems.

The disclosed compounds and/or compositions can be enclosed in multiple or single dose containers. The compounds and/or compositions can also be provided in kits, for example, including component parts that can be assembled for use. For example, one or more of the disclosed compounds may be provided in a lyophilized form and a suitable diluent may be provided as separated components for combination prior to use. In some examples, a kit may include a disclosed compound and a second therapeutic agent for co-administration. The compound and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The active compound is included in the pharmaceutically acceptable carrier in an amount sufficient to exert a therapeutically useful effect in the absence of undesirable side effects on the subject treated. A therapeutically effective concentration may be determined empirically by testing the compounds in known in vitro and in vivo model systems for the treated disorder. In some examples, a therapeutically effective amount of the compound is an amount that lessens or ameliorates at least one symptom of the disorder for which the compound is administered. Typically, the compositions are formulated for single dosage administration. The concentration of active compound in the drug composition will depend on absorption, inactivation, and excretion rates of the active compound, the dosage schedule, and amount administered as well as other factors known to those of skill in the art.

In some examples, about 0.1 mg to 1000 mg of a disclosed compound, a mixture of such compounds, or a physiologically acceptable salt or ester thereof, is compounded with a physiologically acceptable vehicle, carrier, excipient, binder, preservative, stabilizer, flavor, etc., in a unit dosage form. The amount of active substance in those compositions or preparations is such that a suitable dosage in the range indicated is obtained. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. In some examples, the compositions are formulated in a unit dosage form, each dosage containing from about 1 mg to about 1000 mg (for example, about 2 mg to about 500 mg, about 5 mg to 50 mg, about 10 mg to 100 mg, or about 25 mg to 75 mg) of the one or more compounds. In other examples, the unit dosage form includes about 0.1 mg, about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 500 mg, about 600 mg, about 700 mg, about 800 mg, about 900 mg, about 1000 mg, or more of the disclosed compound(s).

The disclosed compounds or compositions may be administered as a single dose, or may be divided into a number of smaller doses to be administered at intervals of time. The therapeutic compositions can be administered in a single dose delivery, by continuous delivery over an extended time period, in a repeated administration protocol (for example, by a multi-daily, daily, weekly, or monthly repeated administration protocol). It is understood that the precise dosage, timing, and duration of treatment is a function of the disease being treated and may be determined empirically using known testing protocols or by extrapolation from in vivo or in vitro test data. It is to be noted that concentrations and dosage values may also vary with the severity of the condition to be alleviated. In addition, it is understood that for a specific subject, dosage regimens may be adjusted over time according to the individual need and the professional judgment of the person administering or supervising the administration of the compositions, and that the concentration ranges set forth herein are exemplary only.

When administered orally as a suspension, these compositions are prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants. If oral administration is desired, the compound is typically provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition. The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily. In some examples, an oral dosage form is administered to the subject 1, 2, 3, 4, or more times daily. In additional examples, the compounds can be administered orally to humans in a dosage range of 1 to 1000 mg/kg body weight in single or divided doses. One illustrative dosage range is 0.1 to 200 mg/kg body weight orally (such as 0.5 to 100 mg/kg body weight orally) in single or divided doses. For oral administration, the compositions may be provided in the form of tablets containing about 1 to 1000 milligrams of the active ingredient, particularly 1, 5, 10, 15, 20, 25, 50, 75, 100, 150, 200, 250, 300, 400, 500, 600, 750, 800, 900, or 1000 milligrams of the active ingredient. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Injectable solutions or suspensions may also be formulated, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers.

The compounds can be administered parenterally, for example, by IV, IM, depo-IM, SC, or depo-SC. When administered parenterally, a therapeutically effective amount of about 0.1 to about 500 mg/day (such as about 1 mg/day to about 100 mg/day, or about 5 mg/day to about 50 mg/day) may be delivered. When a depot formulation is used for injection once a month or once every two weeks, the dose may be about 0.1 mg/day to about 100 mg/day, or a monthly dose of from about 3 mg to about 3000 mg.

The compounds can also be administered sublingually. When given sublingually, the compounds should be given one to four times daily in the amounts described above for IM administration.

The compounds can also be administered intranasally. When given by this route, the appropriate dosage forms are a nasal spray or dry powder. The dosage of the compounds for intranasal administration is the amount described above for IM administration. When administered by nasal aerosol or inhalation, these compositions may be prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents.

The compounds can be administered intrathecally. When given by this route, the appropriate dosage form can be a parenteral dosage form. The dosage of the compounds for intrathecal administration is the amount described above for IM administration.

The compounds can be administered topically. When given by this route, the appropriate dosage form is a cream, ointment, or patch. When administered topically, an illustrative dosage is from about 0.5 mg/day to about 200 mg/day. Because the amount that can be delivered by a patch is limited, two or more patches may be used.

The compounds can be administered rectally by suppository. When administered by suppository, an illustrative therapeutically effective amount may range from about 0.5 mg to about 500 mg. When rectally administered in the form of suppositories, these compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug.

It should be apparent to one skilled in the art that the exact dosage and frequency of administration will depend on the particular compounds administered, the particular condition being treated, the severity of the condition being treated, the age, weight, general physical condition of the particular subject, and other medication the individual may be taking as is well known to administering physicians or other clinicians who are skilled in therapy of retroviral infections, diseases, and associated disorders.

### Examples

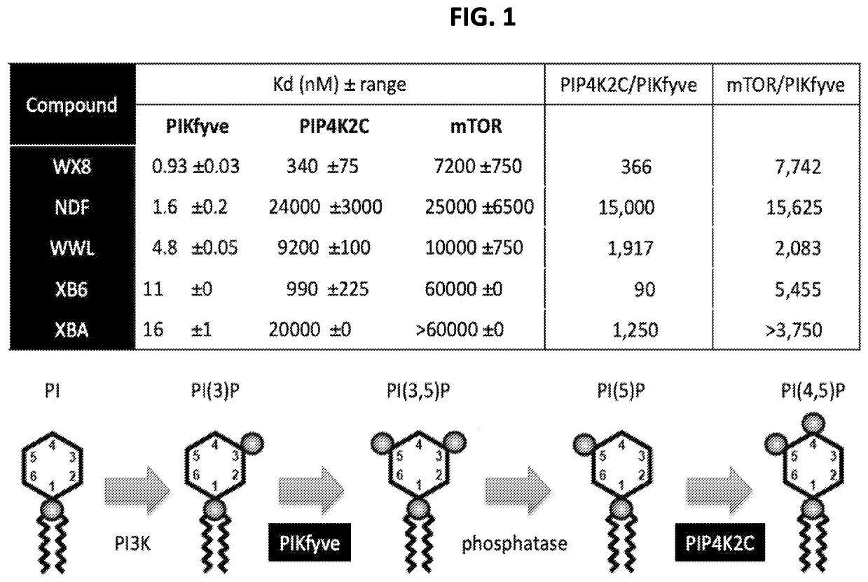

The specificity of the WX8-family for PIKfyve has been demonstrated by their ability to bind specifically to the PIKfyve protein in vitro and in vivo, as well as to mimic the effects of siRNA against PIKfyve mRNA, and by the ability of dominant negative single point mutations in the PIKFYVE gene or disruption of the PIKFYVE gene to nullify the effects of these molecules on cell metabolism. In vitro profiling of human kinases identified the three highest affinity targets as PIKfyve >PIK4K2C>mTOR (FIG. 1). PIKfyve and PIP4K2C are phosphoinositide kinases involved in membrane trafficking. PIKfyve generates PI(3,5)P2 (˜0.1% of total phosphatidylinositol lipids) that serves as both a signaling lipid and the major precursor for PI5P, the substrate for PIP4K2C. mTOR is a phosphoinositide 3-kinase related protein kinase that controls cell growth in response to nutrients and growth factors.

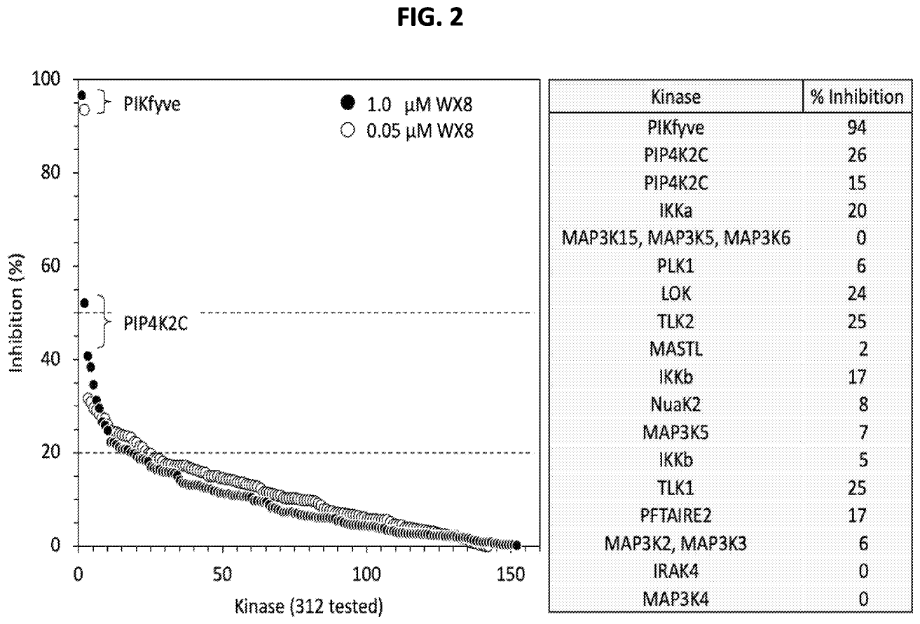

The affinity of WX8-family compounds for PIKfyve varied 400-fold, and their preference for PIKfyve over PIP4K2C varied from 90 to 15,000-fold. The Kd of WX8 was <1 nM for PIKfyve, making its affinity for PIKfyve 366-times greater than for PIP4K2C. This remarkable specificity of WX8 for PIKfyve as the primary target and PIP4K2C as the secondary target was confirmed by analysis of the ability of WX8 to prevent ATP from binding to kinases in autophagy-dependent melanoma A375 cells, a cell line highly sensitive to induction of cell death by the WX8-family (FIG. 2).

The WX8-family of PIKfyve specific inhibitors disrupts multiple events in lysosome homeostasis (Table 1):
prevent lysosome fission without preventing homotypic lysosome fusion, thereby causing cytoplasmic vacuolization (enlarged lysosomes). Cytoplasmic vacuolization is evident within 30 minutes and reversible for up to 24-48 hours (1Vacuoles).
inhibit trafficking of molecules into lysosomes, thereby reducing lysosomal activity
inhibit maturation of lysosome cathepsins, thereby reducing lysosomal activity
disrupt endosome trafficking, thereby reducing cellular uptake of extracellular materials
inhibit cell proliferation (³Proliferation)
disrupt autophagy by preventing heterotypic fusion of lysosomes with autophagosomes (²LC3-II), thereby inducing cell death in autophagy-dependent cancer cells (⁴Viability) under conditions where non-malignant cells remain viable.

TABLE 1
The WX8-family of small molecules are listed in PubChem as either
a compound or a substance (https://www.ncbi.nlm.nih.gov/pubmed/).

| | | Accumulate | | Inhibit | |
|---|---|---|---|---|---|
| | | ¹Vacuoles | ²LC3-II | ³Proliferation | ⁴Viability |
| Compound | PubChem | EC (μM) | EC₅₀ (μM) | IC₅₀ (μM) | IC₅₀ (μM) |
| WX8 | MLS000543798 | 0.01 | 0.3 | 0.2 | 0.35 |
| NDF | MLS000699212 | 0.1 | 1.5 | 1.1 | 0.42 |
| WWL | MLS000703078 | 0.2 | 6.5 | 1.6 | 2.6 |
| XB6 | MLS001167897 | 2 | >10 | 7.4 | 5.3 |
| XBA | MLS001167909 | 4 | 7.5 | 4 | >10 |

The effects of these compounds on U2OS osteosarcoma cells are summarized:
¹Effective concentrations (EC) for inducing vacuoles (enlarged lysosomes) in U2OS osteosarcoma cells within 4 hours.
²Half maximal effective concentration (EC₅₀) for inducing LC3-II (autophagosome) accumulation within 4 to 8 hours.
³Half maximal inhibitory concentration (IC₅₀) for suppressing cell proliferation within 3 days.
⁴The IC₅₀ for reducing cellular ATP levels (viability) within 4 days.

The WX8-Family Selectively Prevents Coronavirus Infection of Cultured Cells WX8 is 100-times more lethal than chloroquine in killing ‘autophagy-addicted’ human melanoma cells, thereby suggesting that one or more of the WX8-family will be more effective and less toxic than chloroquine in preventing SARS-CoV-2 infection of cultured cells and mouse models. The following experimental results confirm this hypothesis.

### 1) The Effects of PIKfyve Inhibitors on Virus Sensitive Host Cells

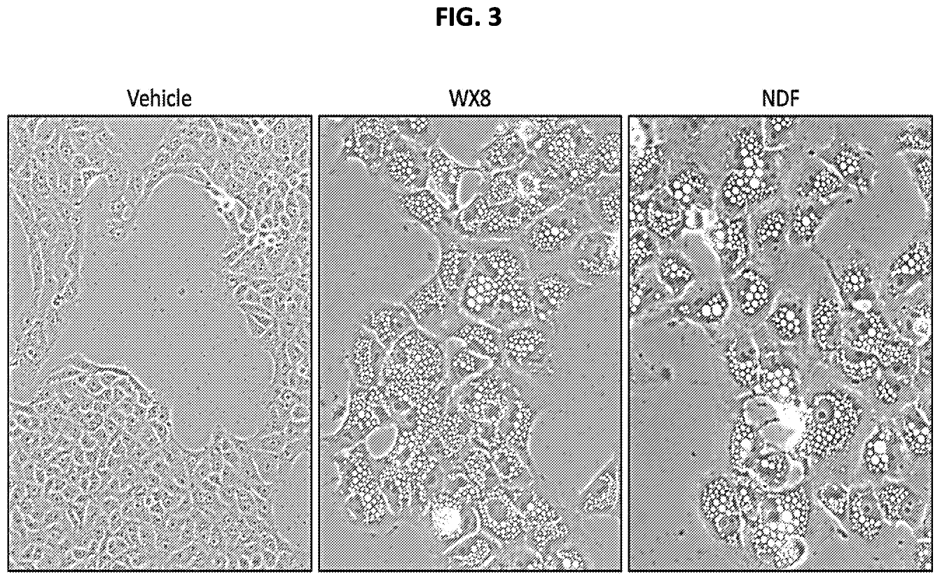

Vero E6 African Green Monkey kidney cells are commonly used for propagating coronaviruses. To determine whether or not Vero E6 cells were sensitive to WX8-family compounds, they were cultured in the presence of WX8, NDF, WWL, or Apilimod. As previously reported for other mammalian cells, Vero E6 cells rapidly accumulated cytoplasmic vacuoles (FIG. 3), the consequence of homotypic lysosome fusion in the absence of lysosome fission.

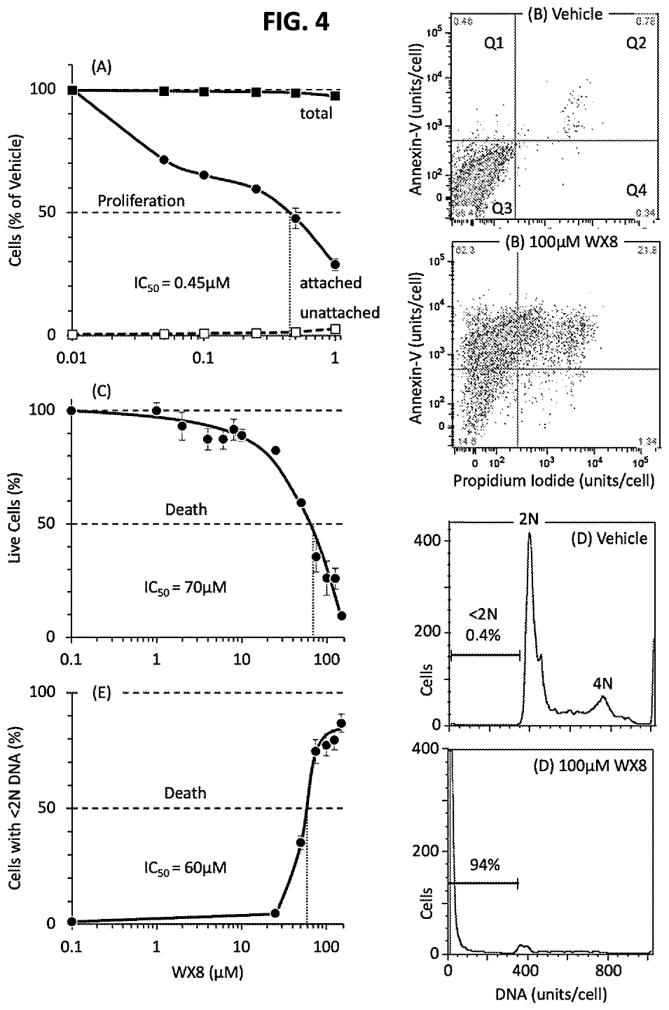

WX8 inhibited Vero E6 cell proliferation with an IC50 of 0.4 μM (FIG. 4A). However, 100-fold higher concentrations of WX8 were required to induce cell death. The fraction of live cells was identified by staining the total cell population (attached plus unattached cells) with both annexin-V and propidium iodide (FIG. 4B). Membrane lipid asymmetry is lost during programmed cell death, and phosphatidylserine becomes exposed on the outer leaflet of the plasma membrane, which allows Annexin-V binding. When the membranes of cells become sufficiently permeabilized, propidium iodide (PI) can enter the cell and bind to DNA. Cells that stain with PI, but not with annexin V, are defined as necrotic cells. WX8 induced Vero E6 cell death with an IC50 of 70 μM (FIG. 4C). Fluorescence activated sorting of cells that were permeabilized and then stained with PI (FIG. 4D) revealed that WX8 induced an accumulation of cells with >2N DNA content (FIG. 4E), a hallmark of cell death. The IP50 for loss of cellular DNA was 60 μM WX8, in excellent agreement with the IP50 for the disappearance of live cells. Similar results were obtained with NDF, WWL, XBA, XB6, and Apilimod and confirmed by the loss of cellular ATP.

2) PIKfyve inhibitors prevent SARS-CoV-2 from infecting virus sensitive host cells without reducing the viability of the host cells.

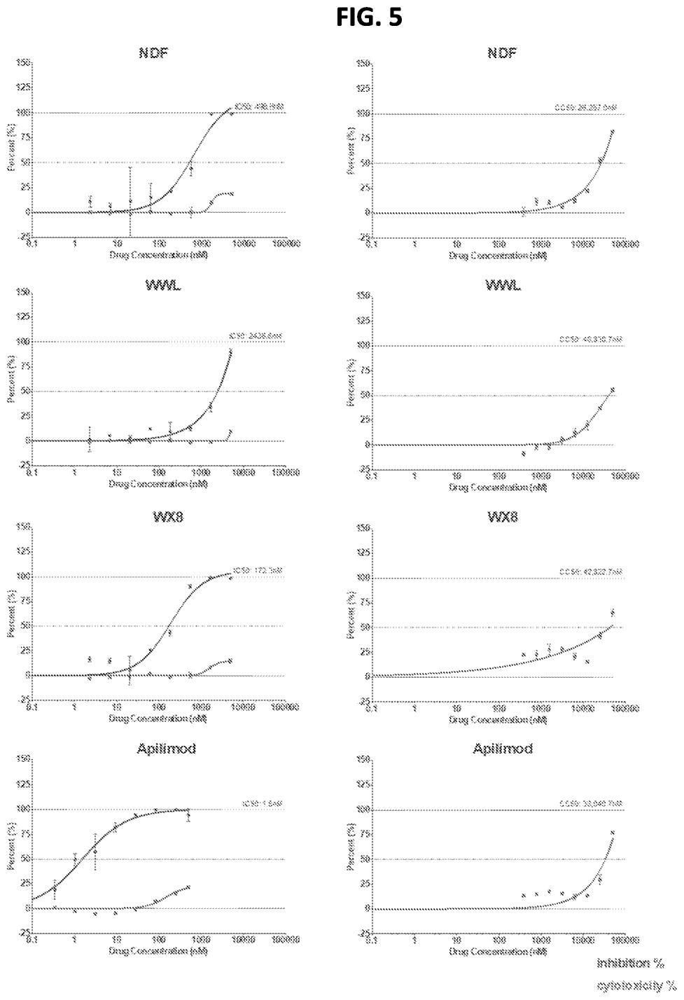

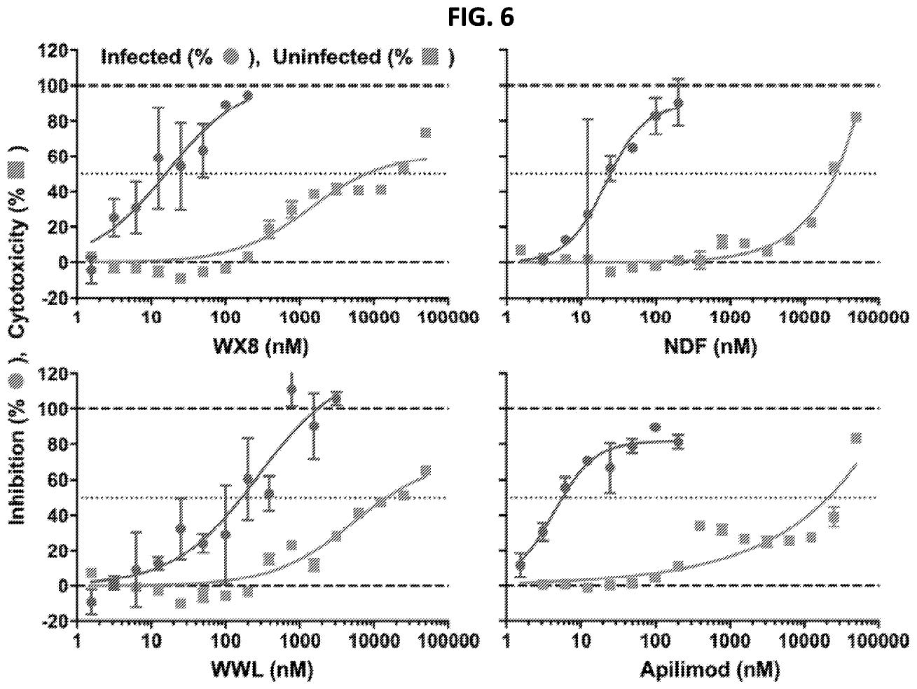

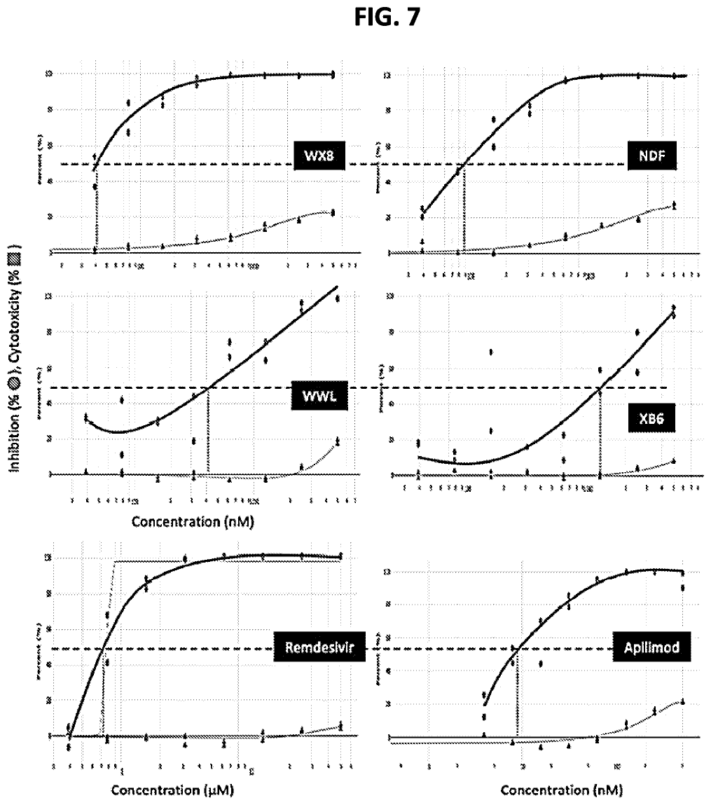

Vero E6 cells were pretreated briefly with each of five members of the WX8-family of PIKfyve inhibitors, and then infected with a low multiplicity of SARS-CoV-2 labeled with green fluorescent protein (GFP). The fraction of cells containing the virus was then quantified as the fraction of cells expressing GFP, and the results plotted as the percentage of inhibition of infection by the drug (FIG. 5, blue circles). See “Drug Screen Data Analysis” for calculations. The IC50 values for preventing SARS-CoV-2 infection ranged from about 2 nM to 2300 nM, depending on the drug (Table 2 below).

The cytotoxicity of each compound was also quantified in parallel by measuring the loss of cellular ATP from uninfected cells (FIG. 5, red squares). See “Drug Screen Data Analysis” for calculations. The IC50 values for cytotoxicity ranged from about 26,000 nM to 42,000 nM, depending on the drug (Table 2).

These values are consistent with those derived from changes in membrane permeability and loss of cellular DNA (FIG. 4). Thus, the WX8-family of PIKfyve inhibitors was able to prevent SARS-CoV-2 from infecting their host cells at concentrations from about 20 to 22,000 times less than concentrations that induced cytotoxicity in uninfected cells.

Together, these results reveal that micromolar concentrations of the five compounds tested here are required to induce metabolic catastrophe (loss of ATP) followed by induction of cell death, whereas nanomolar concentrations can prevent infection by pathogenic coronaviruses.

TABLE 2
Inhibition of SARS-CoV-2 infection of Vero E6 cells

| Compound | IC₅₀ (nM) | CC₅₀ (nM) | CC₅₀/IC₅₀ |
|---|---|---|---|
| WX8 | 172.3 | 42,322 | 245 |
| NDF | 498.6 | 26,287 | 52 |
| WWL | 2438.6 | 40,330 | 16 |
| Apilimod | 1.5 | 33,040 | 22,027 |

The IC₅₀ is the concentration (nM) of each compound that inhibited SARS-CoV-2 infection by 50%. The CC₅₀ is the concentration (nM) of each compound that induced death (cytotoxicity) in 50% of uninfected cells. The ratio CC₅₀/IC₅₀ indicates the efficacy of each compound.

### Materials and Methods Used to Determine the Efficacy and Toxicity of WX8-Family Compounds

1) Cell Culture and virus

Vero E6 cells (ATCC #CRL 1586) were cultured in DMEM (Quality Biological), supplemented with 10% (v/v) fetal bovine serum (Sigma) and 1% (v/v) L-glutamine (2 mM final concentration, Gibco) (Vero Media). Cells were maintained at 37° C. (5% CO₂). Sample of SARS-CoV-2 GFP were generously provided by Dr. Ralph S. Baric (Hou, Y. J., et al., SARS-CoV-2 Reverse Genetics Reveals a Variable Infection Gradient in the Respiratory Tract. Cell, 2020. 182(2): p. 429-446 e14). Stocks were prepared by infection of Vero E6 cells for two days when CPE was starting to become visible. Media were collected and clarified by centrifugation prior to being aliquoted for storage at −80° C. Titer of stock was determined by plaque assay using Vero E6 cells as described previously for MERS-CoV (Coleman, C. M. and M. B. Frieman, Growth and Quantification of MERS-CoV Infection. Curr Protoc Microbiol, 2015. 37: p. 15E 2 1-9). All work with infectious virus was performed in a Biosafety Level 3 laboratory and approved by our Institutional Biosafety Committee. SARS-CoV-2 stock was prepared as previously described for SARS-CoV (Frieman, M., et al., Molecular determinants of severe acute respiratory syndrome coronavirus pathogenesis and virulence in young and aged mouse models of human disease. J Virol, 2012. 86(2): p. 884-97).

### 2) Drug Screen Processing

All drug screens were performed with Vero E6 cells. Cells were plated in clear bottom, black 96-well titer plates (Greiner bio-one, Kremsmunster, Austria) for virus inhibition determination one day prior to infection. Drugs were diluted to a range of concentrations in duplicate. Every compound dilution and control was normalized to contain the same concentration of drug vehicle (e.g., DMSO). Cells were pre-treated with drug for 2 hour (h) at 37° C. (5% CO₂) prior to infection with SARS-CoV-2 GFP at MOI 0.1. In addition to plates that were infected, parallel opaque plates were left uninfected to monitor cytotoxicity of drug alone (“toxicity plates”). Inhibition plates were then incubated at 37° C. (5% CO₂) for 48 hrs, followed by fixation with 4.0% paraformaldehyde, nuclear staining with Hoechst (Invitrogen, Carlsbad, CA), and data acquisition on a Celigo 5-channel Imaging Cytometer (Nexcelom Bioscience, Lawrence, MA). The percent of infected cells was determined for each well based on GFP expression by manual gating using the Celigo software. Compound cytotoxicity was assessed in parallel opaque plates by performing CellTiter-Glo (CTG) assays as per the manufacturer's instruction (Promega, Madison, WI). Luminescence was read on a BioTek Synergy HTX plate reader (BioTek Instruments Inc., Winooski, VT) using the Gen5 software (v7.07, Biotek Instruments Inc., Winooski, VT).

### 3) Drug Screen Data Analysis

Inhibition (% Inhibit) data was normalized according to cell only and virus only controls:

$$\begin{matrix}\% Inhibit ( WT ) = \frac{( drug ) - ( virus only )}{( uninfected ) - ( virus only )} \times 100 \\ \% Inhibit ( GFP ) = ( 1 - \frac{( drug ) - ( cell only )}{( virus only ) - ( cell only )} ) \times 100\end{matrix}$$

Cytotoxicity (% TOX) data was normalized according to cell-only uninfected (cell only) controls and CTG-media-only (blank) controls:

$$\% TOX = ( 1 - \frac{( drug ) - ( media only )}{( cell only ) - ( media only )} ) \times 100$$

Nonlinear regression analysis was performed on the normalized % inhibit and % TOX data and IC50s and CC50s were calculated from fitted curves (log [agonist]versus response−variable slope [four parameters]) (GraphPad Software, LaJolla, CA), as described previously (Dyall, J., et al., In Vitro and In Vivo Activity of Amiodarone Against Ebola Virus. J Infect Dis, 2018. 218(suppl_5): p. S592-S596). Drug dilution points in a given run were excluded from IC50 analysis if the average cytotoxicity was greater than 30% (arbitrary cutoff) across the cytotoxicity replicates for that screen. IC50 or CC50 values extrapolated outside the drug dilution range tested were reported as greater than the highest concentration tested or less than the lowest concentration tested. Selectivity indexes (SI) were also calculated by dividing the CC50 by the IC50.

### Prophetic Example

### Effects of PIKfyve Inhibitors on SARS-CoV-2 Infected and Uninfected Human Lung Cells

We are planning that experiments with Vero E6 cells described above will be reproduced with A549 cells (human lung adenocarcinoma airway epithelial cells) and SABCi cells (human primary small airway epithelial cells), which represent the primary target for SARS-CoV-2 in humans. These experiments are a prelude to experiments with mouse models. After identifying non-toxic levels (highest dose allowed before 30% reduction in cell viability), we anticipate quantifying virus replication as a function of inhibitor concentration. Viral titer, RNA and protein will be quantified over time in the presence and absence of the most efficacious inhibitors and compared to mock infected cells. Based on SARS-CoV-2 results, we anticipate carrying out the same experiments on the related pathogenic viruses SARS-CoV, MERS-CoV, and HCoV-229E.

The effects of these inhibitors on preventing SARS-CoV-2 infection in mice will be evaluated. A mouse adapted SARS-CoV-2 (MA-10) will be used in the Balb/c laboratory mouse model to test these compounds (Leist, S. R., et al., A Mouse-Adapted SARS-CoV-2 Induces Acute Lung Injury and Mortality in Standard Laboratory Mice. Cell, 2020. 183(4): p. 1070-1085 e12). These mice will be treated with selected PIKFYVE inhibitors either before or after infection and the outcome of the infection in the lungs quantified. Pathogenesis can be evaluated in both a prophylactic and therapeutic SARS-CoV-2 infection models. These experiments may determine whether or not the selected PIKFYVE inhibitor affects pathogenesis of SARS-CoV-2 in living animals.

In view of the many possible embodiments to which the principles of the disclosed invention may be applied, it should be recognized that the illustrated embodiments are only preferred examples of the invention and should not be taken as limiting the scope of the invention.

1. A method of inhibiting a SARS-CoV-2 infection in a subject, comprising administering to the subject a therapeutically effective amount of a compound, or a pharmaceutically salt thereof, of formula (I): 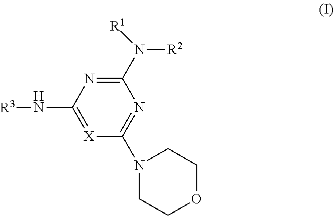
wherein R¹ and R² are independently H, C1-C₆ alkyl, substituted C₁-C₆ alkyl, C₆-C₁₀ aryl, substituted C₆-C₁₀ aryl, or wherein R¹ and R² taken together with the N to which they are attached, form a 5- or 6-membered heterocyclyl ring;
R³ is C₆-C₁₀ aryl, substituted C₆-C₁₀ aryl, R⁴CH═N— wherein R⁴ is C₆-C₁₀ aryl, heteroaryl, or fused bicyclic heteroaryl, or R⁴═CH—NH— wherein R⁴ is C₆-C₁₀ aryl, heteroaryl, or fused bicyclic heteroaryl; and
X is CH or N, provided that the compound of formula I is not vacuolin-1, thereby inhibiting the SARS-CoV-2 infection in the subject.

2. The method of claim 1, further comprising selecting the subject that has the SARS-CoV-2 infection.

3. The method of claim 1, wherein the subject is human.

4. The method of claim 1, wherein the subject has shortness of breath and/or hypoxia.

5. The method of claim 1, wherein the subject has cytokine storm syndrome.

6. The method of claim 1, wherein X is N.

7. The method of claim 1, wherein R¹ and R² taken together with the N to which they are attached, form morpholinyl.

8. The method of claim 1, wherein at least one of R¹ or R² is H.

9. The method of claim 1, wherein one of R¹ or R² is H, and the other of R¹ or R² is phenyl or substituted phenyl.

10. The method of claim 1, wherein R³ is:

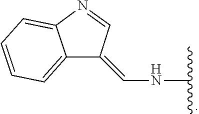 [SMILES: *N/C=C1\C=Nc2ccccc21]

11. The method of claim 1, wherein R³ is phenyl or substituted phenyl.

12. The method of claim 1, wherein R³ is —N═CH—R⁵, wherein R⁵ is phenyl or substituted phenyl.

13. The method of claim 1, wherein the compound is:

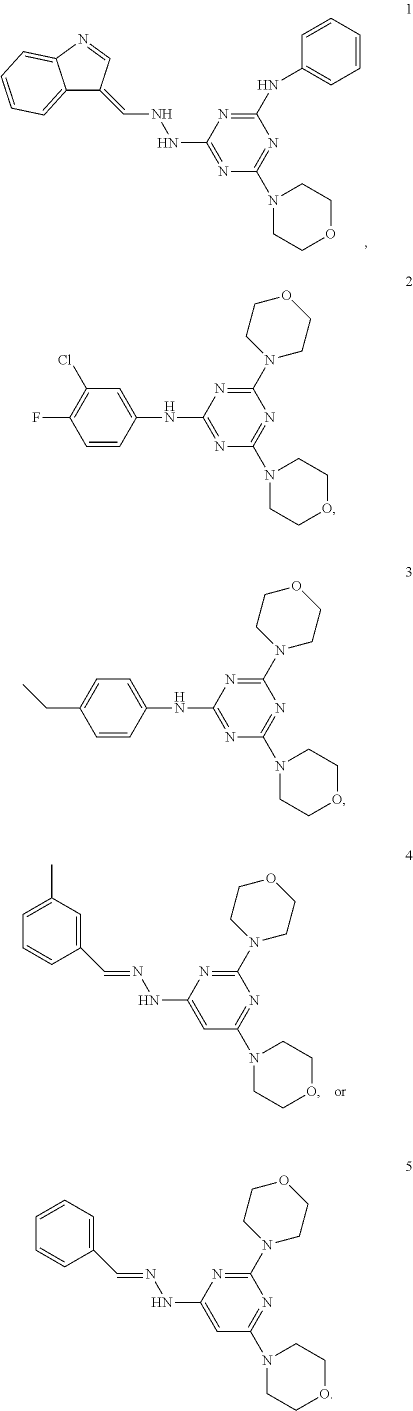 [SMILES: C1=Nc2ccccc2/C1=C/NNc1nc(Nc2ccccc2)nc(N2CCOCC2)n1 ; Fc1ccc(Nc2nc(N3CCOCC3)nc(N3CCOCC3)n2)cc1Cl ; CCc1ccc(Nc2nc(N3CCOCC3)nc(N3CCOCC3)n2)cc1 ; Cc1cccc(/C=N/Nc2cc(N3CCOCC3)nc(N3CCOCC3)n2)c1 ; C(=N\Nc1cc(N2CCOCC2)nc(N2CCOCC2)n1)/c1ccccc1]

14. A pharmaceutical composition comprising a therapeutically effective amount of a compound, or a pharmaceutically salt thereof, of formula (I): 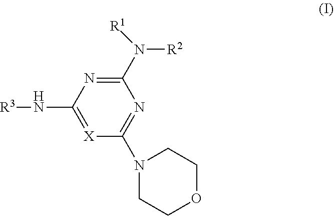
wherein R¹ and R² are independently H, C₁-C₆ alkyl, substituted C₁-C₆ alkyl, C₆-C₁₀ aryl, substituted C₆-C₁₀ aryl, or wherein R¹ and R² taken together with the N to which they are attached, form a 5- or 6-membered heterocyclyl ring;
R³ is C₆-C₁₀ aryl, substituted C₆-C₁₀ aryl, R⁴CH═N— wherein R⁴ is C₆-C₁₀ aryl, heteroaryl, or fused bicyclic heteroaryl, or R⁴═CH—NH— wherein R⁴ is C₆-C₁₀ aryl, heteroaryl, or fused bicyclic heteroaryl; and
X is CH or N, provided that the compound of formula I is not vacuolin-1, for use in treating a subject with a SARS-CoV-2 infection.

15-17. (canceled)

18. The pharmaceutical composition of claim 14, wherein X is N.

19. The pharmaceutical composition of claim 14, wherein R¹ and R² taken together with the N to which they are attached, form morpholinyl.

20. The pharmaceutical composition of claim 14, wherein at least one of R¹ or R² is H.

21. The pharmaceutical composition of claim 14, wherein one of R¹ or R² is H, and the other of R¹ or R² is phenyl or substituted phenyl.

22. The pharmaceutical composition of claim 14, wherein R³ is:

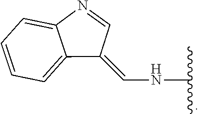 [SMILES: *N/C=C1\C=Nc2ccccc21]

23. The pharmaceutical composition of claim 14, wherein R³ is phenyl or substituted phenyl.

24. The pharmaceutical composition of claim 14, wherein R³ is —N═CH—R⁵, wherein R⁵ is phenyl or substituted phenyl.

25. The pharmaceutical composition of claim 14, wherein the compound is:

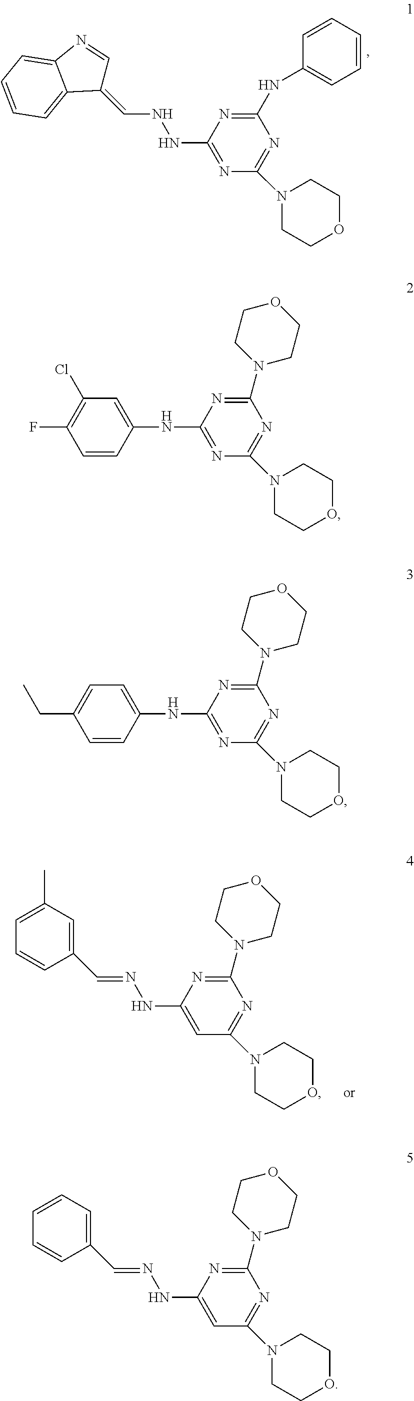 [SMILES: C1=Nc2ccccc2/C1=C/NNc1nc(Nc2ccccc2)nc(N2CCOCC2)n1 ; Fc1ccc(Nc2nc(N3CCOCC3)nc(N3CCOCC3)n2)cc1Cl ; CCc1ccc(Nc2nc(N3CCOCC3)nc(N3CCOCC3)n2)cc1 ; Cc1cccc(/C=N/Nc2cc(N3CCOCC3)nc(N3CCOCC3)n2)c1 ; C(=N\Nc1cc(N2CCOCC2)nc(N2CCOCC2)n1)/c1ccccc1]

26. The method of claim 7, wherein X is N.

27. The pharmaceutical composition of claim 19, wherein X is N.
